(19) United States
(12) Patent Application Publication
(10) Pub. No.: US 2026/0232307 A1
(43) Pub. Date: Aug. 13, 2026

(54) SUTURE ASSEMBLY WITH TUBULAR SHEATH PLEDGET AND METHOD OF USING THE SAME

(71) Applicant: Arthrex, Inc., Naples, FL (US)

(72) Inventors: Corey Stewart, Naples, FL (US); Andrew C. Petry, Naples, FL (US); Patrick Rudersdorf, Golden, CO (US); Gorav Ailawadi, Ann Arbor, MI (US); Marc Gerdisch, Indianapolis, IN (US)

(73) Assignee: Arthrex, Inc., Naples, FL (US)

(21) Appl. No.: 19/533,608

(22) Filed: Feb. 9, 2026

Related U.S. Application Data
(60) Provisional application No. 63/756,479, filed on Feb. 10, 2025.

(51) Int. Cl. A61B 17/04; A61B 17/00

(52) U.S. Cl. CPC A61B 17/0401; A61B 17/0482; A61B 2017/00004; A61B 2017/0406

(57) ABSTRACT

A suture assembly may include a suture having first and second suture ends. A first needle may be coupled to the first suture end. A second needle may be coupled to the second suture end. A tube pledget may be disposed along the suture between the first and second needles. The tube pledget may include a tubular structure defining a lumen. The suture may extend from a first end region of the tubular structure to the first needle and from a second end region of the tubular structure to the second needle.

### CROSS-REFERENCE TO RELATED APPLICATION

This application claims priority under 35 U.S.C. § 119(e) and the benefit of U.S. Provisional Application No. 63/756,479 entitled SUTURE ASSEMBLY WITH TUBULAR SHEATH PLEDGET AND METHOD OF USING THE SAME, filed on Feb. 10, 2025, the entire disclosures of which are incorporated herein by reference.

### TECHNICAL FIELD

The present disclosure generally relates to a suture assembly with a tubular sheath pledget and a method of using the suture assembly for surgical procedures.

### BACKGROUND

Sutures may often be used with pledgets for distributing pressure applied by the suture across tissue. Pledgets can be flat and square, which may result in the pledget twisting and flipping during the surgical procedure. According to some implementations, the suture assembly disclosed herein provides a tube pledget that reduces twisting and flipping of the pledget and twisting of the suture to maximize efficiency of the surgical procedure and provide a better seal against the tissue.

### SUMMARY

Suture assemblies may include a suture having first and second suture ends. A pledget may be coupled to the suture between the first and second suture ends. The suture assembly may also include a needle or other piercing device for carrying the suture ends through tissue.

In certain aspects, a first needle may be coupled to the first suture end, and a second needle may be coupled to the second suture end. A sheath or tube pledget may be disposed along the suture between the first and second needles. The tube pledget may include a tubular structure defining a lumen. The tube pledget may be formed from interlacing strands and may have sealed ends to reduce or prevent fraying of the strands. The suture may extend from a first end region of the tubular structure to the first needle and from a second end region of the tubular structure to the second needle.

The suture may engage the tube pledget in a number of ways. For example, the suture may extend through the lumen and through end openings at opposing ends of the tubular structure. Additionally or alternatively, the tubular structure may include an inner portion configured to engage tissue and an outer portion that define the lumen. The suture may extend through the inner portion of the outer structure proximate to opposing ends of the tubular structure. Further, the suture may extend through both the inner portion and the outer portion proximate to the opposing ends of the tube pledget. The tube pledget and the suture may be engaged in a manner that allows the tube pledget to move or slide along the suture.

The suture assembly may be utilized in a method of performing a surgical procedure, which may include providing a suture assembly including a tube pledget extending along a length of a suture with the suture extending from end regions of the tube pledget; carrying a first suture end through tissue; carrying a second suture end through the tissue spaced from the first suture end; positioning the tube pledget against the tissue between the first and second suture ends; applying a force to the first and second suture ends; automatically unwinding the suture between the tube pledget and the tissue in response to the force applied to the first and second suture ends; and compressing the tube pledget against the tissue in response to the force applied to the first and second suture ends.

These and other features, objects, and advantages of the present disclosure will become apparent upon reading the following description thereof together with reference to the accompanying drawings.

### BRIEF DESCRIPTION OF THE DRAWINGS

FIG. 1 is a side perspective view of a suture assembly with a tube pledget, according to the present disclosure;

FIG. 2A is a side elevational view of a pledget with a tubular structure and a lumen, according to the present disclosure;

FIG. 2B is a side elevational view of a pledget with a tubular structure, a lumen, and a core in the lumen, according to the present disclosure;

FIG. 3 is a schematic diagram of a suture assembly engaging tissue and a valve with a tube pledget of the suture assembly positioned against an under side of the tissue and a suture extending through a lumen and opposing end openings of the tube pledget, according to the present disclosure;

FIG. 4 is a schematic diagram of a suture assembly engaging tissue with a tube pledget of the suture assembly positioned against an under side of the tissue and a suture extending through a tubular structure and along a lumen, according to the present disclosure;

FIG. 5 is a schematic diagram of a suture assembly engaging tissue with a tube pledget of the suture assembly positioned against an under side of the tissue and a suture extending through a tubular structure and transversely across a lumen of the tube pledget, according to the present disclosure;

FIG. 6 is a schematic diagram of a suture assembly engaging tissue with a folded tube pledget of the suture assembly positioned against an under side of the tissue and a suture extending through a tubular structure and transversely across a lumen of the folded tube pledget, according to the present disclosure;

FIG. 7 is a schematic diagram of suture assemblies arranged around an annulus and engaging a replacement valve, according to the present disclosure;

FIG. 8 is a schematic diagram of suture assemblies arranged around an annulus, according to the present disclosure;

FIG. 9 is a schematic diagram of cannulas engaging tissue, according to the present disclosure;

FIG. 10 is representative of a suture assembly with a tube pledget utilized to close a cannulation site, according to the present disclosure;

FIG. 11 is representative of a loading device utilized to load a tube pledget on a suture with the suture extending through a lumen of the tube pledget, according to the present disclosure;

FIG. 12 is representative of a suture with needles piercing a tubular structure of a tube pledget to couple the tube pledget to the suture, according to the present disclosure;

FIG. 13 is a schematic diagram of a tube pledget having multiple tubular structures and multiple lumens, according to the present disclosure;

FIG. 14 is a side perspective view of a tube pledget having a single lumen and a transition section to multiple lumens, according to the present disclosure;

FIG. 15 is a schematic diagram of a suture assembly engaging tissue with a folded tube pledget of the suture assembly on an under side of the tissue and a suture extending through a tubular structure, transversely across a lumen of the tube pledget at end segments, and along the lumen at a centerline, according to the present disclosure; and

FIG. 16 is a method of using a suture assembly with a tube pledget in a surgical procedure, according to the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

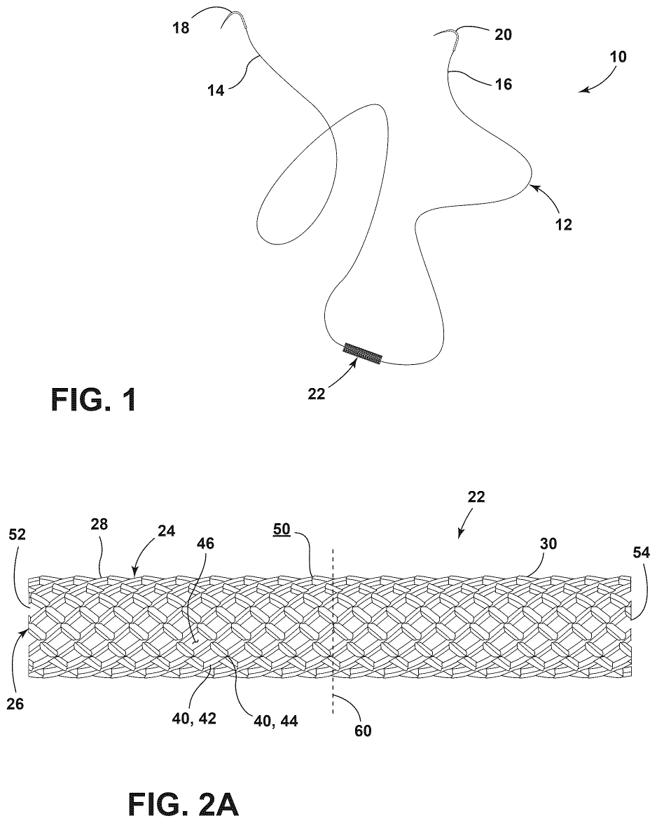

With reference to FIGS. 1-16, reference numeral 10 generally designates a suture assembly 10 that may include a suture 12 having first and second suture ends 14, 16. A first needle 18 may be coupled to the first suture end 14, and a second needle 20 may be coupled to the second suture end 16. A sheath, tubular, or tube pledget 22 may be disposed along suture 12 between the first and second needles 18, 20. The tube pledget 22 may include an outer tubular structure 24 defining a lumen 26 extending along the length thereof. The suture 12 may extend from a first end region 28 of the tubular structure 24 to the first needle 18 and from a second end region 30 of the tubular structure 24 to the second needle 20. The tube pledget 22 may be configured to move along the length of the suture 12 to position the pledget 22 against tissue T.

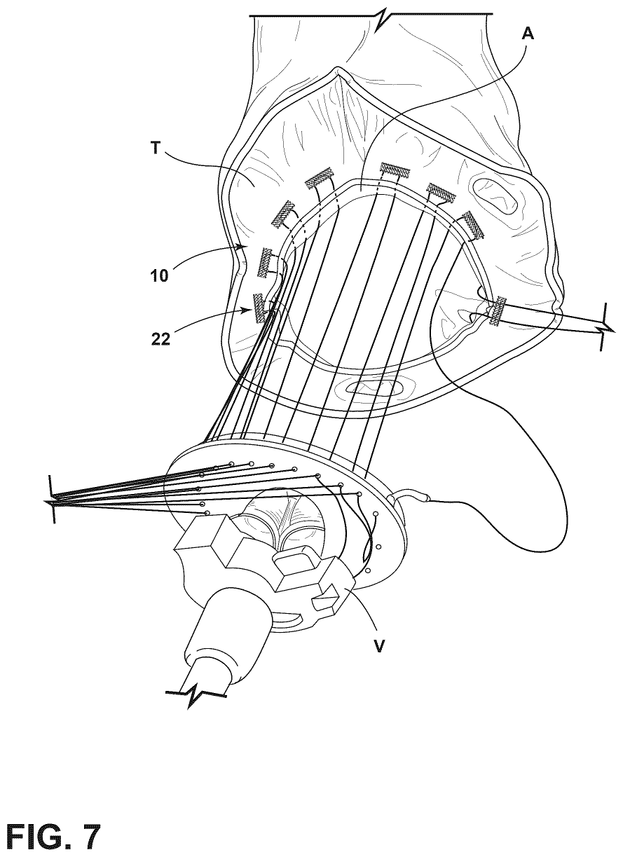

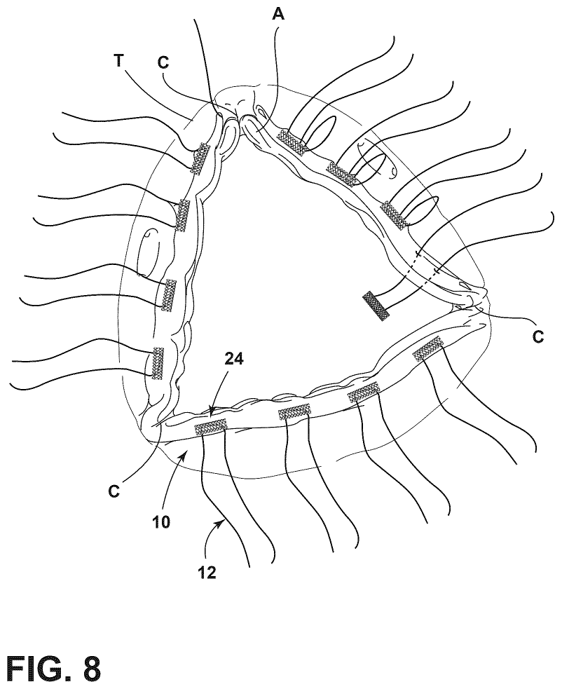

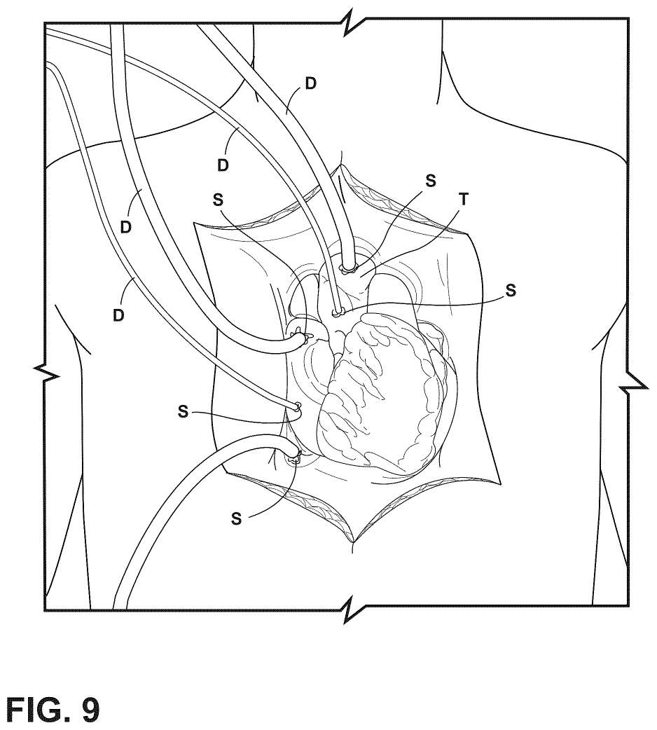

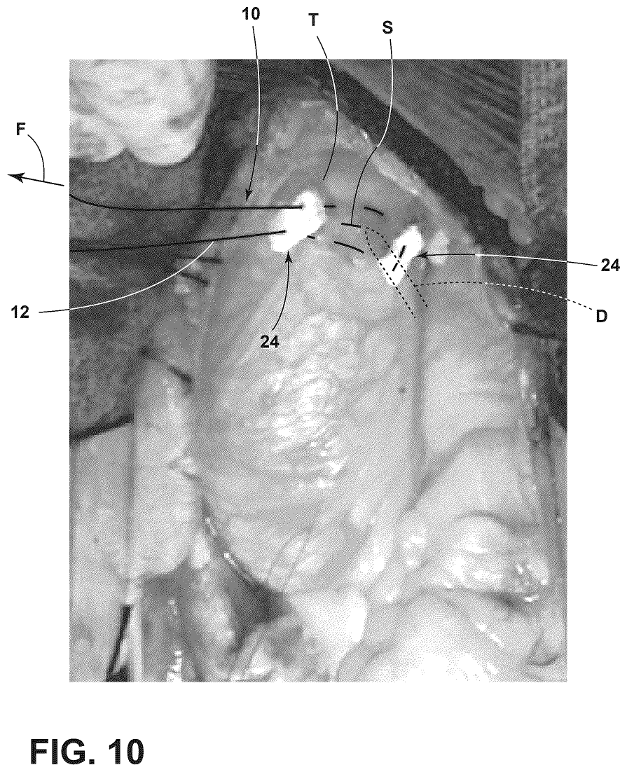

The suture assembly 10 can be utilized for a variety of procedures, including valve replacement procedures (see FIGS. 7 and 8) and closing arterial cannulation sites S (see FIGS. 9 and 10). The tube pledget 22 may generally form an extension of the suture 12 and can compress against the tissue T to assist with forming a seal, such as, for example, between the tissue T and a replacement valve V or at the cannulation site S. The suture assembly 10 with the tube pledget 22 may be used in a variety of fields, including urology, gynecology, thoracic surgery, cardiac surgery, plastic surgery, reconstructive surgery, etc. Exemplary cardiac procedures include valve replacements, valve procedures (e.g., aortic, mitral, pulmonic, tricuspid), cannulation, aortic anastomoses, left ventricle restoration, septal defects, commando operations, chordal repair during mitral repair/tricuspid repair, repair for bleeding sites (e.g., cannulation bleeding site, vessel closure bleeding such as in the atrium, aortic closure line, etc.), etc. The suture assembly 10 may be used in any procedure where a suture 12 and a pledget 22 can be used, including those where the native tissue may be weaker. The suture 12 may extend from the end regions 28, 30 of the tube pledget 22, which may reduce or prevent twisting and/or flipping of the tube pledget 22 and the suture 12 during the procedure(s).

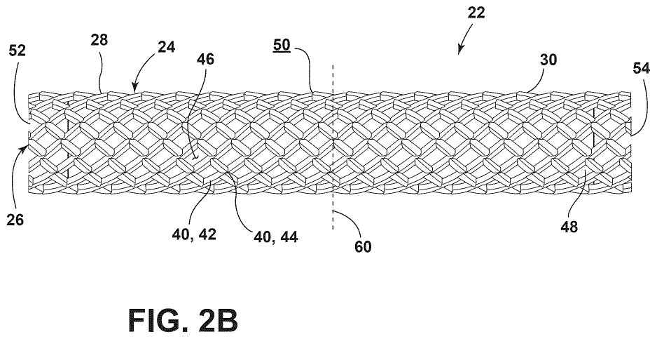

Referring to FIGS. 1 and 2A, the tube pledget 22 is generally a mesh-like component that can assist with distributing pressure from the suture 12 along a greater surface area of the tissue T. The pledget 22 may be constructed of intertwined or interlaced yarns or strands 40 and may be constructed using various techniques, such as weaving, braiding, knitting, or otherwise interlacing the strands 40. The interlacing configuration may allow retracting and expanding movement of the tube pledget 22 and may also allow the suture 12 to be passed therethrough. Additionally, the interlacing strands 40 may allow the tube pledget 22 to be moved along the length of the suture 12 as described herein.

The tubular structure 24 is generally a cylindrical shape with a hollow interior (e.g., the lumen 26). Though described as tubular, it is understood that the cross-section of the tubular structures 24 may be elliptical, rectangular, or other profile shapes along the length to enclose and define the lumen 26. The tubular structure 24 may have consistent or varying cross-sectional shape(s) and/or may have a diameter that may remain constant or vary along the length. For example, the end regions 28, 30 may taper, or a portion of the tubular structure may be elliptical/flattened. The tubular structure 24 may be formed by one or more layers of interlinked strands 40 that may be manufactured through multiple techniques in an interlaced configuration. The tubular structure 24 may be formed by navigating the corresponding strands 40 through complex interlinked paths along which bobbing carriers may dispense the strands 40 in coordination. In this way, the proportions, thickness, and/or materials associated with the tubular structure 24 may vary along the length thereof, which may be advantageous for different uses of the tube pledget 22 in different surgical procedures.

The tubular structure 24 may be constructed using multiple carriers, each carrying at least one strand 40 to arrange the interlacing strands 40 in various patterns. The tubular structure 24 may be formed from between about eight and about sixty-four carriers and, consequently, between about eight and about sixty-four strands 40. In a non-limiting example, the tubular structure 24 may be formed using sixteen carriers. The strands 40 or yarns forming the tubular structure 24 may travel along the interlinked paths, forming extended figure “8” or lemniscate shapes, loops, helical shapes, crisscrossing patterns, etc. Each strand 40 may follow a predefined path that provides a connection to form the tubular structure. The strands 40 may be in patterns, such as one-over-one (e.g., one strand 40 over one strand 40), one-over-two, two-over-one, or two-over-two patterns. While extending along the interlinked paths, the strands 40 also form the cylindrical, tubular structure 24 and the lumen 26.

In the illustrated configuration in FIG. 2A, a first set 42 of the interlacing strands 40 may extend in a helical pattern in a first direction (e.g., clockwise) and may extend parallel to one another. A second set 44 of the interlacing strands 40 may extend in a helical pattern in a second opposing direction (e.g., counterclockwise) and may extend parallel to one another. The strands 40 in each set 42, 44 may be arranged at substantially equal intervals to form a crossing, mesh pattern between the two sets 42, 44 of strands 40. The second set 44 of strands 40 may be woven around the first set 42 (e.g., over/under) or may be inserted through the first set 42 (e.g., the second set 44 intersects through the strands 40 of the first set 42). The sets 42, 44 of strands 40 can be arranged in crossing or overlapping helical patterns, forming openings 46 between adjacent ones of the first set 42 of strands 40 and adjacent ones of the second set 44 of strands 40.

The pattern of the openings 46 may be based on the pattern of the strands 40. For example, the openings 46 may be arranged in a helical pattern based on the helical patterns of the strands 40. The openings 46 may allow some movement of the pledget 22 in the length direction. In this way, the helical patterns may slightly deform to elongate, increasing the length of the tubular structure 24 and decreasing the width/diameter, or to compress, decreasing the length of the tubular structure 24 and increasing the width/diameter. The space between the strands 40 provided by the openings 46 may assist in maintaining the overall structure of the tube pledget 22, as well as allow some degree of deformation, which may be advantageous for transferring pressure from the suture 12 along the length of the tube pledget 22 and compressing the tube pledget 22 against the tissue T. The openings 46 may also provide locations for inserting the suture 12 therethrough as described herein.

The tube pledget 22 may be constructed of, for example, coreless suture strands 40. Coreless sutures 12 may be advantageous for providing a flatter, more pliable tubular structure 24. However, sutures 12 with cores and/or jackets may be utilized without departing from the teachings herein. The strands 40 may be constructed of polyester, polytetrafluoroethylene (PTFE), polypropylene, silk, or a combination thereof. The strands 40 and, consequently, the pledget 22, may be absorbable or non-absorbable. In certain aspects, the pledgets 22 may be RTF pledgets 22. The strands 40 may be monofilament and/or braided multifilament. Monofilament strands 40 may have a thickness between 10 microns and 200 microns. The strands 40 may be constructed in different colors to provide a visual indication during the surgical procedure. Additionally or alternatively, a single tube pledget 22 may be constructed to have different segments of different colors to provide visual indicators or identifiers during the surgical procedure or for assisting in coupling the tube pledget 22 to the suture 12 as described herein.

The strands 40 may also have different thicknesses or weights for different procedures. Further, different types of strands 40 may be utilized in a single tube pledget 22 to balance different properties of the strands 40. In certain aspects, at least some of the strands 40 may be 16×830 dtex strands 40 or yarns. The strands 40 may form the tube pledget 22 having one or more braid densities (PPI). The strands 40 may be arranged for the tube pledget 22 to have a braid PPI between about 10 PPI and about 200 PPI, such as between about 24 PPI and about 32 PPI.

The properties of the strands 40 may affect the properties of the overall pledget 22. Wall thickness, braiding technique, and yarn properties may all be adjusted to adjust the overall pledget 22 and the properties thereof. These properties may include material density/thickness, overall structure diameter, braid density, size of strands 40, etc. For example, the material density/thickness may be between about 10 dtex and about 1000 dtex. The strands 40 and, consequently, the pledget 22 may be constructed of a variety of materials, such as, but not limited to, polyester, polypropylene, nylon, polybutester, silk, plain gut, chromic gut, polyglactin, poliglecaprone, PDS (polydioxanone), polydioxanone polyglycolic acid (e.g., Dexon®), vicryl, PTFE, monocryl, barbed suture (any material), stainless steel wire, and other similar materials. A number of these materials may be absorbable, allowing the pledget 22 to be absorbable. The absorbable material may allow the suture assembly 10 or components thereof to break down naturally via, for example, hydrolysis or enzymatic degradation, which may reduce a need for secondary access to remove the suture assembly 10 and allow the treated anatomy to settle or heal without potential interference or obstruction associated with the interaction between the tissue and the suture assembly. An absorbable pledget 22 may accomplish the immediate goals of dispersing force, enhancing hemostasis, and reinforcing fragile tissue while potentially decreasing the long-term negative impact (e.g., scar tissue, infection risk, etc.) of having foreign material internally or externally within/on the heart or other vascular structures.

Additionally, aspects of the overall pledget 22 may also be changed. For example, the wall thickness may be about 1 mm or less. The overall structure diameter may be in a range between about 0.2 mm and about 4 mm. Further, a single pledget 22 may include strands 40 of different sizes/thicknesses, such as by interlacing larger and smaller yarns. This may adjust rigidity (e.g., larger yarns may increase rigidity), porosity, and/or flexibility of the pledget 22. The pledget 22 may have sufficient rigidity or thickness to hold shape and apply pressure, while sufficient flexibility to move along the suture 12 and provide a buttressing effect.

Further, as illustrated in FIG. 2B, the pledget 22 may include a core 48 at least partially filling the lumen 26, which can also affect properties of the pledget 22. The core 48 may have different constructions or materials to adjust the properties of the pledget 22. For example, the core 48 may be a monofilament core or may be a stiffer braided core with a softer outer jacket (e.g., providing a stiff yet “fluffy” feel). The tube pledget 22 may have the same properties along the length thereof or, alternatively, may include different properties, such as one or more diameters, braid PPIs, etc., along the length thereof, without departing from the teachings herein.

Referring still to FIGS. 1-2B, the tube pledget 22 may have a variety of sizes for different procedures. In certain aspects, the tube pledget 22 may have a length between about 2 mm and about 36 mm. In cardiothoracic examples, the tube pledget 22 may generally be between about 3 mm and about 15 mm. In cannulation examples, the pledget 22 may have a length between about 2 mm and about 7 mm. The tube pledget 22 may have an outer width or diameter of between about 1 mm and about 5 mm. The tube pledget 22 may be constructed in a manner that may form the tubular structure 24 with any practicable a predefined length and width.

Additionally, the tube pledget 22 may be constructed to end in a manner that reduces or prevents fraying of the strands 40 or loosening of the interlacing pattern. For example, the ends of the pledget 22 may be sealed. This sealing may be accomplished via thermal sealing to melt the strands 40 or fibers to form smooth, rounded edged. This may include heat cutting, laser cutting, ultrasonic cutting, or similar processes. The ends may also be sealed via adhesive end bonding. This may be accomplished with, for example, cyanoacrylate or epoxy (or similar materials) and/or ultraviolet cure. Further, the ends may be sealed with a coating. The pledget 22 may be coated with a selected material, such as a medical-grade polymer (absorbable or non-absorbable), which may include antimicrobial, silicone, PTFE, polycaprolactone (PCL), and/or similar materials. This process may allow the fibers to fuse along the length of the suture/strand 40, which can then be cut in manufacturing or in the operating room.

Moreover, the sealing at the ends of the pledget 22 may remain narrow to reduce or minimize the “hardened” effect on the strands 40 that may be caused by the sealing process. For example, the securing distance at the ends of the pledget 22 may be between about 0.01 mm and about 2 mm, such as about 0.125 mm. This may seal the ends of the pledget 22 to reduce fraying, while reducing the hardness of the pledget 22. The softer pledget 22 may be advantageous for different procedures and can reduce roughness at the edges that may engage tissue.

In a specific non-limiting example for cardiothoracic procedures, the tube pledget 22 may have a length between about 6 mm and about 8 mm and a width/diameter of between about 1.5 mm and about 2.5 mm. The thickness of the tubular structure 24 may be less than about 0.5 mm (e.g., from an outer surface 50 of the tubular structure 24 to an inner surface forming the lumen 26). Accordingly, the tube pledget 22 may be elongated, having a greater length than width to reduce twisting/flipping of the tube pledget 22 while also having a thickness that balances distributing pressure from the suture 12 with minimizing overall profile size for being implanted in the patient.

In the illustrated configuration in FIGS. 2A and 2B, the tubular structure 24 may have a rounded profile along an outer circumference/surface 50, such that the tubular structure 24 forms a circular or oblong cross-sectional shape. In such examples, the tubular structure 24 generally has a similar width and height. In additional or alternative configurations, the tube pledget 22 may be more oblong, compressed, or flattened. In such configurations, the height may be different than the width of the tubular structure 24. For example, the tubular structure 24 may be flattened to have a lesser height and a greater width, which may provide increased surface area for engaging the tissue T. The height may be decreased for the outer portion of the tubular structure 24 to abut the inner portion of the tubular structure 24, which may form a “tape-like” tube pledget 22 with a compressed lumen 26 extending therethrough.

Referring still to FIGS. 1-2B, the strands 40 forming the tubular structure 24 may also define the lumen 26 extending along the length of the tube pledget 22. The strands 40 may be arranged in the pattern(s) that form the lumen 26. In other words, the winding or helical pattern may allow the strands 40 to extend around and form the lumen 26. Generally, the lumen 26 has a consistent width/diameter along the length thereof. However, configurations with a narrowing or widening lumen 26 and/or with multiple lumens 26 may be utilized as described herein.

The lumen 26 may generally be an open channel that extends along the length of the tubular structure 24. The tubular structure 24 may define end openings 52, 54 at the opposing distal ends of the end regions 28, 30, respectively, to provide access to the lumen 26. The end openings 52, 54 may generally be coaxial with and a same size as the lumen 26. Alternatively, the end openings 52, 54 may be offset and/or differently sized than the cross-section of the lumen 26, which may assist with guiding the tube pledget 22 along the suture 12. For example, smaller end openings 52, 54 may reduce movement of the suture 12 within the lumen 26.

The tube pledget 22 may be positioned on and extend along the length of the suture 12 between the first and second suture ends 14, 16, which may be at least initially coupled to the first and second needles 18, 20. By extending along the length of the suture 12, the tube pledget 22 may form an extension of or a thickened portion of the suture 12 that is movable relative to the suture 12. The tube pledget 22 is configured to move along the length of the suture 12. The tube pledget 22 may be freely movable along the length and, in certain aspects, may rotate about the suture 12. In other words, the tube pledget 22 may not be fixed in a location along the suture 12. The movable aspect of the tube pledget 22 along the length of the suture 12 may be advantageous for maneuvering the suture 12 and the tube pledget 22 relative to the anatomy of the patient during the procedure. The movable relationship may also reduce twisting of the suture 12 between the anatomy and the tube pledget 22 during the procedure and reduce folding or the pledget against the tissue T.

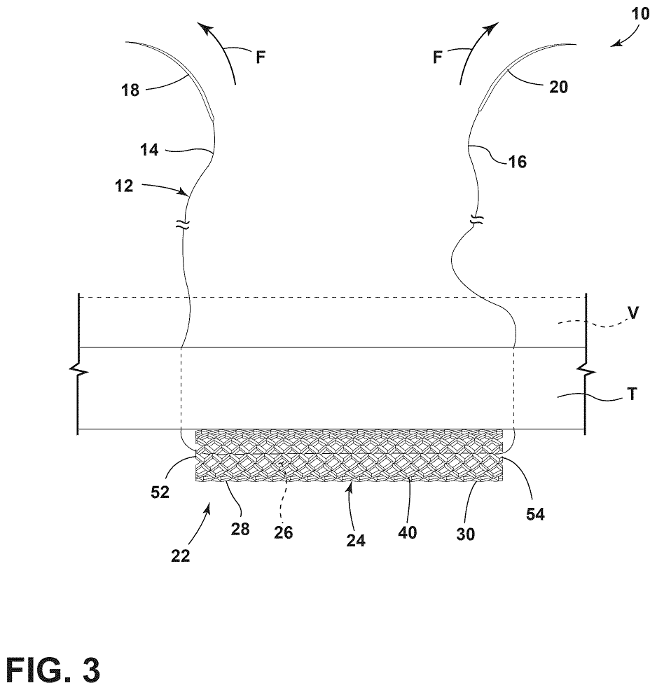

Referring to FIGS. 3-6, the suture 12 is configured to extend through the tube pledget 22. In certain aspects, the suture 12 may extend through the lumen 26, which may be along the length of the lumen 26 and/or transversely across the lumen 26. The suture 12 may also extend parallel with the lumen 26. The suture 12 may extend through (e.g., along or across) the lumen 26 and from the end regions 28, 30 of the tubular structure 24. The end regions 28, 30 may be at any location between a centerline 60 and the distal ends of the tubular structure 24 forming the end openings 52, 54. In various aspects, the suture 12 may extend from the tubular structure 24 at the distal ends or locations closer to the distal ends than the centerline 60. Accordingly, the suture 12 may extend along the length of the tubular structure 24, allowing the tube pledget 22 to form a thickened portion or extension of the suture 12 and reducing excess material of the tube pledget 22 that can be inadvertently folded.

In the illustrated configuration in FIG. 3, the suture 12 extends along the entire length of the lumen 26. The suture 12 extends through the lumen 26 and through the end openings 52, 54. Accordingly, in this configuration, the tubular structure 24 may extend circumferentially around the suture 12, and the suture 12 may not extend through the tubular structure 24. Stated differently, the tubular structure 24 may be free of the suture 12 extending therethrough. The tubular structure 24 may freely move along the length of the suture 12 and may rotate about the suture 12. The suture 12 may extend: from the first needle 18, through the first end opening 52, through the lumen 26, through the second end opening 54, and to the second needle 20. This configuration may allow the most movement of the tube pledget 22 relative to the suture 12 without piercing the tubular structure 24. This configuration may also maximize the reduction in excess material of the pledget 22 by extending through the end openings 52, 54. Additionally, this configuration may maximize the pressure distribution from the suture 12 by extending across the greatest length of the pledget 22.

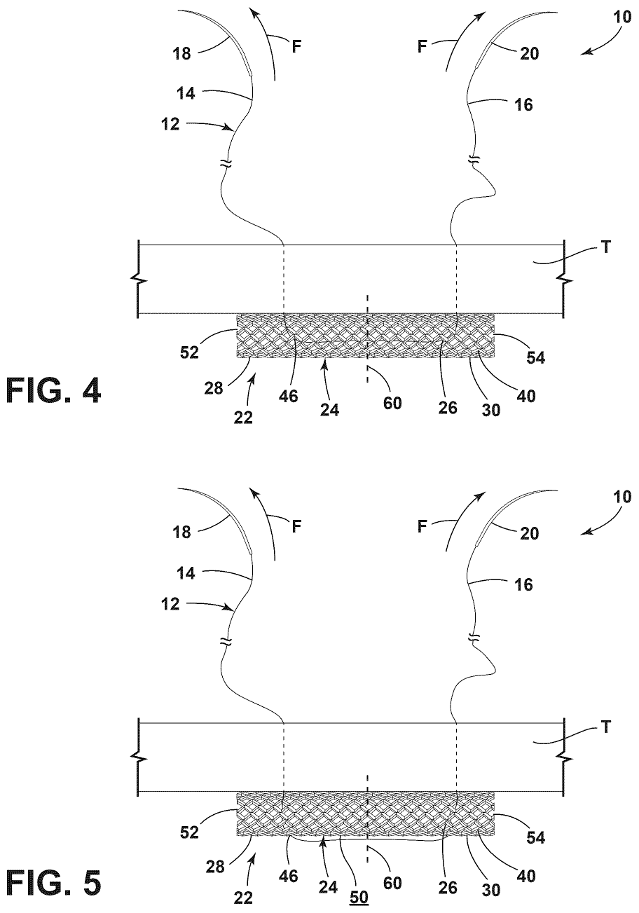

In the configuration illustrated in FIG. 4, the suture 12 extends along a substantial portion of the lumen 26 but may not extend through the end openings 52, 54. Instead, the suture 12 may extend through the end regions 28, 30 of the tubular structure 24, such as through openings 46 between the strands 40. Typically, the suture 12 may extend through the tubular structure 24 proximate to the end openings 52, 54 or closer to the end openings 52, 54 than the centerline 60. The relationship with the suture 12 extending from the end regions 28, 30 of the pledget may reduce the excess material of the tube pledget 22 that can twist or fold around the suture 12. Additionally, the extension from the end regions 28, 30 may assist with unwinding or reducing twisting of the suture 12 in response to an applied force F.

In this configuration, the suture 12 extends through the tubular structure 24 in two locations. The two locations are typically on a same side or form a line that is generally parallel with the lumen 26. Typically, the two locations may be aligned with one another rather than offset along the circumference of the tube pledget 22 to reduce twisting of the pledget 22 in response to the applied force F. The suture 12 may extend through the tubular structure 24 at two locations along a portion of the tubular structure 24 configured to engage the anatomy of the patient (i.e., an inner portion). The suture ends 14, 16 may extend in a same direction through the tubular structure 24. At the two locations where the suture 12 extends through the tubular structure 24, the suture 12 may extend through openings 46 defined by the interlacing strands 40, and between these two locations, the suture 12 may extend through the lumen 26. In sum, the suture 12 may extend: from the first needle 18, through the tubular structure 24 at a first location proximate to the first end opening 52, through the lumen 26, through the tubular structure 24 at a second location proximate to the second end opening 54, and to the second needle 20.

This configuration may allow the tube pledget 22 to move along the length of the suture 12 while the tubular structure 24 may be fixed in a rotational direction (i.e., the tube pledget 22 may not be configured to rotate about the suture 12 due to the suture 12 extending through the tubular structure 24). This configuration may be advantageous for reducing movement of the pledget 22 along the suture 12 as the openings 46 in the tubular structure 24 are smaller than the end openings 52, 54. The suture 12 extending through the tubular structure 24 may assist with pulling or compressing the tube pledget 22 against the tissue T in response to the force F applied to the suture ends 14, 16.

In the configuration illustrated in FIG. 5, the suture 12 extends transversely through the lumen 26 and may not extend along a substantial portion of the length of the lumen 26. The suture 12 may extend through the tubular structure 24 at two locations in the first end region 28 and two locations in the second end region 30. In this way, the suture 12 may extend through two locations of the inner portion of the tubular structure 24 that engage the tissue T and two locations of an opposing outer portion of the tubular structure 24, where the inner portion and the outer portion collectively define the lumen 26.

In the first end region 28, the suture 12 may extend through the tubular structure 24 and into the lumen 26, across the lumen 26, and out of the lumen 26 through the tubular structure 24. Generally, these two locations in the first end region 28 may be opposing locations and aligned with one another such that the suture 12 extends perpendicularly to the length of the lumen 26. In the second end region 30, the suture 12 may extend in the opposing direction through the tubular structure 24 and into the lumen 26, across the lumen 26, and out of the lumen 26 through the tubular structure 24 for the suture end 14, 16 to extend from a same side of the tube pledget 22 (e.g., the inner portion). These two locations in the second end region 30 may also be aligned with the suture 12 extending perpendicularly to the lumen 26.

Between the pass-through locations at the outer portion in the first and second end regions 28, 30, the suture 12 may extend along the outer surface 50 of the tubular structure 24 and along the length of the tubular structure 24. The suture 12 may extend generally parallel with the lumen 26. The suture 12 may generally not extend along the circumference of the tubular structure 24 to reduce twisting of the tube pledget 22.

The inner and outer through locations may be generally diametrically opposed from one another, and the locations at each of the first and second end regions 28, 30 may be aligned for the portions of the suture 12 extending transversely across the lumen 26 to be parallel with one another and perpendicular to the length of the lumen 26. Alternatively, the suture 12 portions may be angled depending on the surgical procedure. Additionally, the suture ends 14, 16 generally extend parallel and/or coplanar with one another from the tube pledget 22 to apply a more even force on the ends of the tube pledget 22 and, consequently, on the tissue T.

This relationship between the suture 12 and the tube pledget 22 may allow for the tubular structure 24 to be more compressed against the tissue T and provide two layers of the pledget 22 between the suture 12 and the tissue T. This configuration may assist with providing greater compression with the pledget 22. The tension of the suture 12 may be drawn against anatomy such that portions of the suture 12 passing through the tubular structure 24 and the lumen 26 may compress the tube pledget 22. As a result, the overlapping inner and outer portions of the tubular structure 24 may collapse and compress against the anatomy, thereby providing a stronger suture 12 engagement. In this configuration, the tube pledget 22 may be configured to move along the length of the suture 12 but may not be configured to rotate about the suture 12 due to the suture extending through the tubular structure 24.

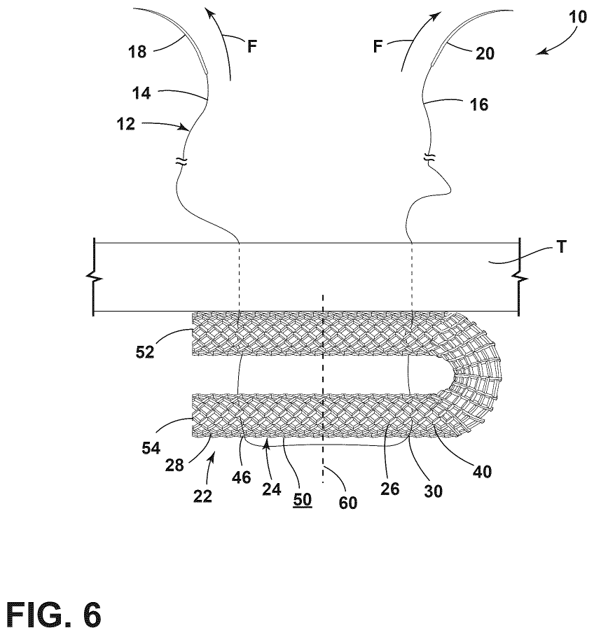

In the configuration illustrated in FIG. 6, the tube pledget 22 may be folded. In certain aspects, the tube pledget 22 may be folded once to form a general “U” or “C” shape with the end regions 28, 30 disposed adjacent to one another. It is also contemplated that the tube pledget 22 may be folded more than once, such as to form an “S” shape, “zigzag” shape, etc. The suture 12 may generally extend through the tubular structure and transversely across the lumen 26, similar to the configuration illustrated in FIG. 5.

For example, as illustrated in FIG. 6, at the first end region 28 abutting the tissue T, the suture 12 may extend through the tubular structure 24, transversely across the lumen 26, and then extend out of the tubular structure 24. The suture 12 may then extend through the second end region 30 abutting the first end region 28, extending again through the tubular structure 24, transversely across the lumen 26, and out of the tubular structure 24. The four pass-through locations in the end regions 28, 30 may generally retain the tube pledget 22 in the folded configuration.

The suture 12 may then extend along the outer surface 50 of the tubular structure 24 and toward the centerline 60. Proximate to the centerline, the suture 12 may extend through the tube pledget 22. The suture 12 may extend through the tubular structure 24, transversely across the lumen 26, and through the tubular structure 24 on a first side of the centerline 60. The suture 12 may then extend through the tubular structure 24, transversely across the lumen 26, and through the tubular structure 24 on a second side of the centerline 60. The folded tube pledget 22 may include two layers of the tubular structure 24, and the suture 12 may extend through each layer.

In this regard, the suture generally extends through the first layer abutting the tissue T, through the second layer abutting the first layer, along the outer surface 50, through the second layer, and through the first layer. Typically, the suture 12 extends transversely across the lumen 26 but may extend partially through the lumen 26 without departing from the teachings herein (see FIG. 15). The folded tube pledget 22 may provide additional density and depth on the under side of the tissue T and may form a buttress against the tissue T for the suture assembly 10. Additionally, the folded pledget 22 may be able to slide or adjust along the suture 12 and may also reduce twisting or flipping of the tube pledget 22 and the suture 12, similar to the configurations illustrated in FIGS. 3-5. Moreover, the tube pledget 22 in the folded configuration may provide increased compression of the pledget 22 against the tissue T.

The pledget 22 may be constructed in a manner that allows the needles 18, 20 to penetrate through the wall of the pledget, as illustrated in FIGS. 5 and 6. In this way, the suture 12 may be inserted through the pledget 22 and across the lumen 26 at any location along the length of the pledget 22. This provides increased flexibility for the location of the suture 12 relative to the pledget 22 and in-operation assembly of the suture assembly 10.

Referring again to FIGS. 3-6, as described herein, the suture ends 14, 16 may extend in the same direction from the tube pledget 22. However, other orientations or configurations may be utilized without departing from the teachings herein. For example, the suture 12 may extend into the lumen 26 from the inner portion of the tubular structure 24 and out of the lumen 26 through the outer portion of the tubular structure 24.

Each of these configurations between the suture 12 and the tube pledget 22 may reduce excess material of the pledget 22 to reduce the folding of the pledget 22 during the procedure. The material may be reduced at the ends of the tube pledget 22 as the suture 12 extends from the end regions 28, 30. Additionally, the cylindrical shape of the tube pledget 22 may reduce material along the width of the pledget 22 as the tube pledget 22 forms an extension of the suture 12. The reduction in excess material may minimize the folding of the pledget 22 during the procedure, which can result in reduced efficiency of the pledget 22 in closing the opening in the tissue T or distributing the force F applied by the suture 12.

Without the benefit of the tube pledget 22 and the relationship between the tube pledget 22 and the suture 12, the pledget may inadvertently twist, fold, or turn against the anatomy of the patient. The inadvertently folded pledget can affect the outcome of the procedure. For example, in a valve replacement example, the pledget may twist, turn, or fold in an annulus A of the valve, which can affect hemodynamics and valve leakage due to less security in how the valve V is seated with the tissue T. This may result in an increase in the size of the implanted material in the patient and may reduce the fluid-tight seal provided by the suture 12. Further, this issue may also decrease the efficiency of the procedure, resulting from the medical professional taking time to unfold the inadvertently folded pledget 22. The relationships of the suture 12 with the tube pledget 22 described herein may reduce or prevent this folding, twisting, or turning to provide a better seal and tighter compression by the suture 12 while increasing accuracy in positioning of implanted devices and reducing procedure time.

The tube pledget 22 is configured to be positioned against the anatomy to distribute the force F applied by the suture 12 and assist with providing a tight seal. The tube pledget 22 being elongated and narrow (e.g., the generally cylindrical shape) may reduce twisting of the suture 12, as well as folding of the pledget 22. For example, as a pulling force F is applied to the needles 18, 20 on an outer side of the anatomy to pull the suture ends 14, 16 through the anatomy, the force F may cause the suture 12 between the under side of the anatomy and the tube pledget 22 to unwind until the limbs of the suture 12 are extending generally parallel to one another. With the force F being transferred from the suture 12 to the end regions 28, 30, the tube pledget 22 may automatically rotate in response to the applied force F to unwind the limbs of the suture 12. This may be advantageous for automatically reducing twisting and tangling of the suture 12, as well as reducing the length of suture 12 on the under side of the anatomy. The automatic unwinding may increase the efficiency of the procedure and may result in a better seal provided by the suture assembly 10.

Further, the cylindrical tube shape and the stiffness of the strands 40 may reduce flipping or folding of the tube pledget 22, which can affect the position of the tube pledget 22 against the anatomy. In this regard, the suture 12 extending from the end regions 28, 30 may result in the applied force F from the suture 12 being applied closer to the ends of the elongated tube pledget 22, which may retain the orientation/configuration of the tube pledget 22 to reduce folding of the tube pledget 22. Further, tube pledget 22 may form an extension of the suture 12 to reduce movement of the pledget 22 independently of the suture 12. For example, in the configuration where the suture 12 pierces or is threaded through the end regions 28, 30 (see FIGS. 4 and 5), there is less excess material to contribute to the pledget 22 flipping and folding around the suture 12 and/or against the anatomy. In the configuration where the suture 12 extends through the end openings 52, 54 (see FIG. 3), rotation of the pledget 22 about the suture 12 may not significantly change the profile/shape of the pledget 22 and, therefore, may not significantly change how the pledget is pressed against the anatomy.

The tube pledget 22 may also be configured to move along the length of the suture 12, which may assist with positioning the tube pledget 22 against the anatomy. Additionally, the tube pledget 22 may be configured to move relative to the suture 12, such as by elongating or compressing. This may assist with distributing pressure from the suture 12 against the anatomy, as well as compressing the tube pledget 22 against the anatomy with reduced bunching. However, the pledget 22 may be fixed to the suture 12 without departing from the teachings herein. The elongated and narrow configuration of the tube pledget 22 may reduce the twisting of the tube pledget 22 and the twisting of the suture 12.

Referring still to FIGS. 3-6, the different relationships between the suture 12 and the tube pledget 22 may provide different advantages. For example, with the suture 12 passing through the lumen 26 and the end openings 52, 54, such as the configuration illustrated in FIG. 3, the tube pledget 22 may extend circumferentially around the suture 12, which may reduce or prevent any flipping or twisting that affects the relationship between the pledget and the anatomy. The tube pledget 22 may rotate about the suture 12 and maintain the same general profile to compress against the anatomy. The suture 12 extending partially through the lumen 26 and through the tubular structure 24, such as the configuration illustrated in FIG. 4, may reduce rotation of the tube pledget 22 to reduce movement during the procedure. Additionally, the tube pledget 22 may move less freely along the length of the suture 12 to properly position the tube pledget 22 due to the smaller openings 46 through which the suture 12 extends compared to the end openings 52, 54. The configurations in FIGS. 3 and 4 may be pre-assembled and pre-packaged together in the illustrated arrangement, allowing the sutures assembly 10 to be unpackaged and ready-to-use in a sterile environment.

The suture 12 extending through the tubular structure 24 and transversely across the lumen 26, such as the configuration illustrated in FIGS. 5 and 6, may provide greater compression of the tube pledget 22 against the tissue T. The suture 12 may compress the tube pledget 22, flattening the tube pledget 22 and reducing the size/height of the lumen 26 as the tube pledget 22 is pulled against the tissue T. This may provide a cushion under the tissue T for greater distribution of the pressure from the suture 12. Additionally, this may reduce the size of the tube pledget 22. Additionally, the folded tube pledget 22 illustrated in FIG. 6 may provide a thicker cushion to form a buttress against the tissue T. The configurations in FIGS. 3 and 4 may be pre-assembled and pre-packaged or may be assembled by the surgical team prior to a procedure.

Each of the relationships may allow the tube pledget 22 to be moved along the length of the suture 12, which may assist with positioning the suture assembly 10 for the procedure. Further, the movement may assist with reducing the twisting of the suture 12 and the tube pledget 22. Moreover, the suture 12 extending from the end regions 28, 30 of the tube pledget 22 may allow the tube pledget 22 to function as an extension of the suture 12. This configuration can assist with reducing twisting of the suture 12 and flipping or folding of the pledget 22, providing a greater seal with the suture assembly 10.

The suture 12 may be any practicable suture 12 for the selected procedure. For example, the suture 12 may be Prolene®, polyethylene, Ethibond®, polyester, polypropylene, nylon, polybutester, silk, plain gut, chromic gut, polyglactin, poliglecaprone, PDS (polydioxanone), polydioxanone polyglycolic acid (e.g., Dexon®), vicryl, PTFE, monocryl, barbed suture (any material), stainless steel wire, and other similar materials. etc. The suture 12 may be between 0-0 in size and 5-0 in size. The size and configuration of the suture 12 may depend on the procedure. For example, for cannulation, a 3-0 or 4-0 Prolene ® suture 12 or a 3-0 Ethibond® suture 12 may be used. For chordal repair during mitral/tricuspid repairs, the suture 12 may be a CV4 or CV5 PTFE suture 12. The suture 12 may also be a round suture, a flat suture (e.g., SutureTape™), have flat segments, and/or have round segments. The suture 12 may be selected for the configuration of the pledget 22 and/or the selected procedure. The suture 12 may be secured via one or more securing features, which may be: knots, COR-KNOT®, slip knots, crimps, grommets, clips, knotless mechanisms (e.g., shuttling process, a one-way locking mechanism with locking barbs, etc.), suture-tensioning tools, a zip-like or zipper tensioning features, locking ferrules or collars (e.g., with single direction barbs), or other components and/or processes that retain the sutures 12 under tension.

In knotless suture 12 examples, the knotless suture 12 constructs may be integrally formed from the structure 24 or strands 40 forming the pledget 22 and may be formed in a common manufacturing step of a textile manufacturing process (e.g., braiding, weaving, etc.). The pledget 22 with knotless sutures 12 may allow for fewer sutures 12 with increased compression to reduce paravalvular leaks. Typically, the knotless sutures 12 may be part of a suture assembly having a guide or shuttle suture 12 and a repair suture 12. The repair and the shuttle suture 12 may extend through a locking feature, which may be built into the pledget 22 and/or positioned in the lumen 26. The shuttle suture 12 may guide the repair suture 12 through the locking feature to retain the repair suture 12 under tension in response to a shuttling process. The shuttle suture 12 may be removed from the suture assembly 10, leaving the repair suture 12 looped through the tissue under tension. The locking feature may be a spliced segment of the repair suture 12 (e.g., the repair suture 12 is pulled through itself) or a grommet. The knotless suture 12 may allow the suture 12 to extend across a greater surface area of the tissue to increase compression.

Referring to FIGS. 7-10, the suture assembly 10 may be utilized for connecting devices or implants to soft tissue T/muscle, such as the replacement valve V in FIGS. 7 and 8, or for closing openings in soft tissue T, such as the cannulation sites S in FIGS. 9 and 10. For example, as illustrated in FIGS. 7 and 8, the suture assembly 10 may be utilized for heart valve replacement or heart valve repairs, such as a mitral valve replacement or an aortic valve replacement. The replacement valve V may be a mechanical valve or may be a tissue-based valve. The suture assembly 10 may assist with defining a fluid-tight or blood-tight seal between the tissue T and the replacement valve V.

Using the aortic valve replacement as an example, the annulus A extends around leaflets of the aortic valve, and the leaflets are removed from the patient. The suture assembly 10 may be configured to draw the replacement valve V into the patient to implant and replace the valve that was removed from the patient. The replacement valve V can be brought to engage and be compressed against the tissue T. The replacement valve V is generally sized to correspond with the size of the annulus A into which the replacement valve V is being sewn. For example, if the opening of the tube has a 27 mm diameter, then a 27 mm replacement valve V may be used to reduce or prevent any gaps outside of the replacement valve V.

Multiple suture assemblies 10 may be utilized to securely place the replacement valve V. For example, between about 12 and 17 suture assemblies 10 may be utilized. The suture assemblies 10 can be spaced around the annulus A to secure the replacement valve V to the tissue T. The suture assemblies 10 may be utilized initially at the commissures C where the leaflets meet. The suture assemblies 10 may be tightened in sequence and may be secured through one or more techniques (e.g., knotted, crimped, secured with the knotless configuration, etc.).

The tube pledget 22 may be arranged along an inner surface of the tissue T. The suture assembly 10 may include the two needles 18, 20 provided to pass the suture ends 14, 16 through the tissue T and the replacement valve V from a first side (e.g., the under side) to an opposing second side (i.e., an outer side). The first needle 18 can carry the first suture end 14 through the tissue T and the replacement valve V. Similarly, the second needle 20 can be inserted through the tissue T and the replacement valve V. The second needle 20 is generally inserted through the tissue T and the replacement valve V in a location spaced from the first needle 18. The spacing may generally align with the length of the tube pledget 22, such that the suture 12 may extend from the end regions 28, 30 of the tube pledget 22 and directly into the tissue T, which can maximize the pressure distribution provided by the pledget 22 and reduce flipping or folding. After passing the needles 18, 20 through the tissue T and the replacement valve V, the suture ends 14, 16 may be disposed on an opposing side of the tissue T and the replacement valve V relative to the tube pledget 22.

The use of the two needles 18, 20 can assist with strengthening the engagement between the tissue T and the replacement valve V to provide a better seal and compression of the replacement valve V into the tissue T. For example, with the aortic valve replacement, there is generally a high gradient of blood flow that moves through the aortic valve into the body of the patient to be able to pump blood through the extremities of the patient. The two pass through locations of the suture assembly 10 through the tissue T and replacement valve V may assist in providing a blood-tight seal between the tissue T and the replacement valve V with greater compression therebetween.

The pulling force F can be applied to the needles 18, 20 and/or the suture ends 14, 16 to pull the length of the suture 12 through the tissue T and the replacement valve V, as well as pull the tube pledget 22 against the tissue T. The relationship with the suture 12 extending from the end regions 28, 30 of the tube pledget 22 may allow the suture 12 to automatically unwind as the suture 12 is pulled through the tissue T and the replacement valve V. In this way, in response to the pulling force F, the suture 12 may unwind for the limbs to extend parallel from the tube pledget 22 and reduce or prevent a length of the suture 12 from being caught between the tube pledget 22 and the tissue T. The configuration of the tube pledget 22 may reduce the twisting and flipping of the tube pledget 22 during the procedure to position the tube pledget 22 directly against the tissue T, increasing the surface area of the pledget 22 engaging the tissue T and providing a better seal with the suture assembly 10.

During the procedure, the position of the tube pledget 22 may be changed along the length of the suture 12 to provide the selected or predefined length of the suture 12 on the opposing side of the replacement valve V. Further, the length of the suture ends 14, 16 may be continually adjusted by applying the pulling force F to one suture end 14, 16 to move the suture 12 through the tissue T and the tube pledget 22. This may be advantageous for more properly positioning the suture assembly 10 during the procedure and for securing the suture 12. In various aspects, the suture 12 may be tied or knotted to secure the suture 12. Additionally or alternatively, a crimp may be utilized to secure the suture 12. In additional non-limiting examples, the suture assembly 10 may be a knotless suture assembly 10 that may use a shuttling process to form a knotless stitch configuration.

Referring to FIGS. 9 and 10, the suture assembly 10 may also be utilized for closing an opening in the soft tissue T, such as a cannulation site S. In various procedures, a cannula or other device D may be inserted into the soft tissue T. Upon removal of the cannula D, an opening remains in the soft tissue T, which can be closed by the suture assembly 10 with the tube pledget 22. For example, the suture assembly 10 may be positioned at the cannulation site S. The suture assembly 10 may be arranged around the inserted cannula D or may be positioned around the cannulation site S upon removal of the cannula D. In certain aspects, the suture assembly 10 may form an area for a cannulation incision prior to cannula D insertion.

The cannulation incision may be made in the tissue T, and the cannula D may be inserted through the incision. In certain aspects, the suture assembly 10 may be arranged around the inserted cannula D and may be tightened around the cannula D to assist with securing the cannula D to the tissue T. The tube pledget 22 may be arranged on an outside of the tissue T proximate to the cannulation site S. The needles 18, 20 may be utilized to pass the suture 12 through the tissue T multiple times to form a purse string around the cannulation site S. Accordingly, the suture 12 may extend from the end regions 28, 30 of the tube pledget 22 and around the perimeter of the cannulation site S to an opposing side of the cannulation site S. The suture ends 14, 16 and the needles 18, 20 may be arranged on the opposing side of the cannulation site S compared to the tube pledget 22. An additional tube pledget 22 may be utilized proximate to the suture ends 14, 16 and opposite the first tube pledget 22 to further distribute pressure from the force F applied to the suture 12.

The tube pledget 22 may assist with reducing tearing or bleeding in response to applying force F to the suture ends 14, 16 to close the cannulation site S. The force F may be applied to the suture ends 14, 16 to cinch the tissue T around the cannula D. Upon removal of the cannula D, the pulling force F may be applied to the suture ends 14, 16, which may be transferred along the length of the suture 12 to cinch the tissue T and close the cannulation site S. The suture 12 may be secured (e.g., with a knot, crimp, knotless securement, etc.) to hold the cannulation site S closed. The suture 12 extending around the cannulation site S may result in the tissue T being drawn toward the center of the incision to close the incision utilizing a single suture assembly 10. For closing the cannulation site S, the tube pledget 22 may generally be arranged on the outer side of the tissue T. However, for certain incisions, it is also contemplated that the suture assembly 10 may be utilized with the tube pledget 22 arranged against the underside of the tissue T (e.g., similar to the valve replacement arrangement) without departing from the teachings herein.

The suture assembly 10 may be pre-assembled for the medical team for use in a sterile setting. In such examples, the suture assembly 10 may be pre-packaged with the suture 12 extending through the tube pledget 22. The suture assembly 10 may also be provided with the needles 18, 20 attached to the suture ends 14, 16. The pre-assembled suture assembly 10 may be removed from the packaging and immediately utilized in the procedure.

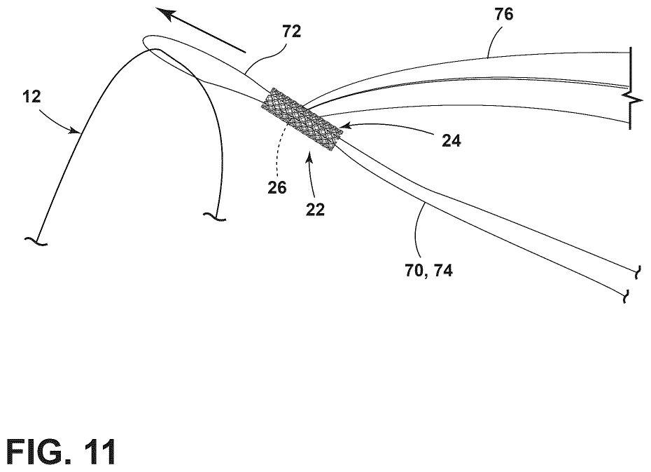

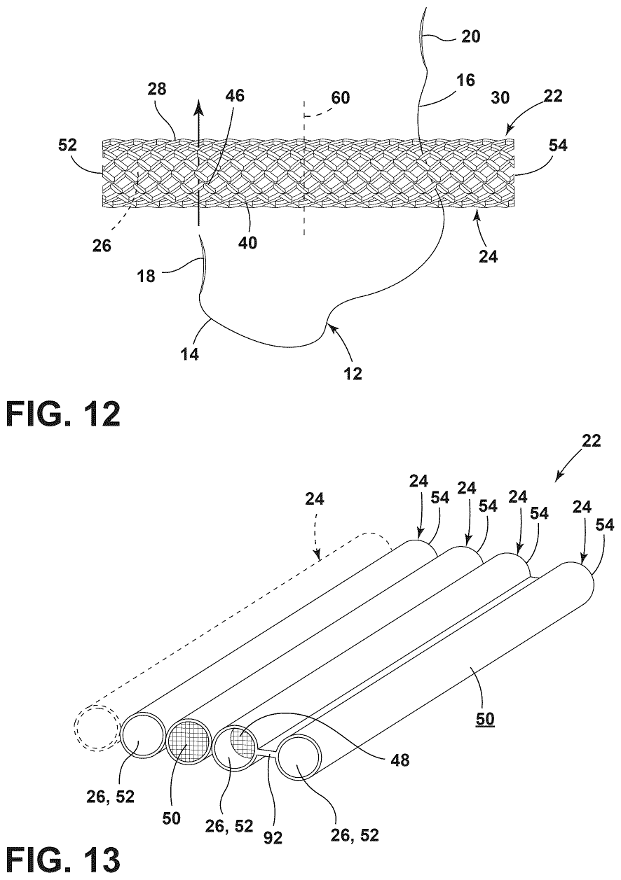

Referring now to FIGS. 11 and 12, in certain aspects, the surgical team may assemble the suture assembly 10. For example, as illustrated in FIG. 11, the surgical team may position the tube pledget 22 with the suture 12 extending through the lumen 26 to form the suture assembly 10 illustrated in FIG. 3. A loading device 70, such as a MegaLoader or other loop-based loading device, may be utilized, and the tube pledget 22 may be loaded onto the loading device 70. The loading device 70 may include a loop, such as a nylon monofilament loop (e.g., welded into a loop) that can be pre-loaded with the suture assembly 10. For example, the loading device 70 may extend through the lumen 26 with an end loop 72 of the loading device 70 on an opposing side of the tube pledget 22 compared to a grasping location 74. The suture 12 may be inserted through the end loop 72. The medical professional may slide or otherwise move the tube pledget 22 away from the grasping location 74, over the end loop 72, and onto the suture 12. Typically, the tube pledget 22 may be moved using forceps 76 or a similar device to move the tube pledget 22 over the loading device 70 and the suture 12.

The suture 12 may be folded, extending through the lumen 26 twice and be engaged around the end loop 72 of the loading device 70. The medical professional may apply a pulling force F on a portion of the suture 12 to pull one of the limbs from the lumen 26, leaving the suture 12 extending through the lumen 26 and the end openings 52, 54 with the suture ends 14, 16 on opposing sides of the tube pledget 22. The loading device 70 may be disengaged from the suture 12. In certain aspects, the suture assembly 10 may be pre-loaded or pre-coupled to the loading device 70. Accordingly, the loading device 70 with the suture assembly 10 may be pre-packaged to be opened and ready-to-use in a sterile setting. The needles 18, 20 may then be attached to the suture ends 14, 16 or, alternatively, the suture assembly 10 with the suture 12 and the tube pledget 22 may be utilized with an insertion device.

Referring to FIG. 12, the medical or surgical team may also position the tube pledget 22 on the suture 12 to form the suture assembly 10 illustrated in FIG. 5. The surgical team may attach the needles 18, 20 or, alternatively, the suture 12 may be pre-assembled with the needles 18, 20. The surgical team may then insert the first needle 18 through the first end region 28 of the tube pledget 22. The first needle 18 can carry the first suture end 14 through the tube pledget 22. The first needle 18 and the first suture end 14 may pass through the tube pledget 22 at two locations and transversely across the lumen 26.

The second needle 20 may be inserted through the second end region 30. The second needle 20 can carry the second suture end 16 through the tube pledget 22. The second needle 20 and the second suture end 16 may pass through the tube pledget 22 at two locations and transversely across the lumen 26. Generally, the portions of the suture 12 extending through the tube pledget 22 may extend parallel with one another. The length of the needles 18, 20 may assist with inserting the suture 12 in a manner where the limbs extend parallel to one another and perpendicular to the longitudinal extent of the tube pledget 22. The needles 18, 20 may generally be inserted through the tube pledget 22 closer to the end openings 52, 54 than the centerline 60 to reduce twisting and flipping of the suture 12 and the tube pledget 22. The tube pledget 22 may then be moved along the length of the suture 12 between the needles 18, 20 and utilized in the procedure.

The first and second needles 18, 20 may be utilized to carry the suture 12 through the tube pledget 22 and/or the tissue T before being removed from the suture assembly 10. It is contemplated that certain devices may be utilized in lieu of the needles 18, 20 for carrying the suture ends 14, 16 through the tissue T and/or the replacement valve V without departing from the teachings herein. In such examples, the device may function similarly to the needles 18, 20 for engaging the suture ends 14, 16 during the procedure, then disengaging from the suture 12 for the suture 12 securing process (e.g., tying, shuttling, etc.). In other words, the needles 18, 20 may not be included in the suture assembly 10. Rather, the insertion devices can be utilized with the suture assembly 10 that includes the suture 12 and the tube pledget 22, which may be advantageous when the tube pledget 22 is applied to the suture 12 by the medical professional or if the needles 18, 20 have been otherwise removed from the suture assembly 10.

The insertion device may be a suture passer, which may include a distal jaw with at least one movable jaw member and a movable needle. The jaw may be utilized for holding and moving tissue T. The jaw may also support the movable needle, which can be selectively engaged with the suture 12 to be passed through the tissue T. In certain aspects, the user may load one or both suture ends 14, 16 on the needle of the suture passer. Additionally or alternatively, the suture passer may be configured to automatically reload the second suture end 16 of the suture 12 onto the needle. The suture passer may be used for passing the suture 12 without using the attached needles 18, 20.

In additional non-limiting examples, the insertion device may be an implant device for placing the suture assembly 10 in the tissue T. The implant device may carry or house one or both suture ends 14, 16 of the suture 12 and the tube pledget 22. A needle tip of the implant device can be inserted through the tissue T and/or the replacement valve V, and the user can deploy the first suture end 14 of the suture assembly 10. The user can then adjust the implant device to deploy the needle tip to carry the second suture end 16 through the tissue T and/or the replacement valve V at a second location. Use of the implant device may be advantageous when the surgical team assembles the suture 12 with the tube pledget 22.

Referring again to FIGS. 1-12, the tube pledget 22 may be utilized with different types of sutures 12. In this way, the suture 12 in the suture assembly 10 may have a variety of configurations or sizes that are suitable for the selected procedure. The suture 12 may be tape-like, compressed, rounded, rounded that compresses flat, etc. Additionally, different portions of the sutures 12 may have different configurations. For example, the suture 12 may have a tape-like portion that may become pledget-like. Further, the suture 12 may also include a bifurcated or spliced section for use in the knotless securement of the suture 12. In various aspects, the suture 12 may be constructed of polyester or polyethylene (PTFE), which may be more inert and stronger than polyester. The increased strength of PTFE may be advantageous for applying the pulling force F to the suture 12.

Referring to FIGS. 13-15, the suture assembly 10 may include different configurations of the tube pledget 22. For example, the tube pledget 22 may define one or more sizes and/or one or more lumens 26. In this regard, the pledget 22 includes various constructs that may provide for one or more interconnected features, such as multiple tubular structures 24 that may form adjacent lumens 26 extending along the length thereof. The adjacent tubular structures 24 may be interlinked such that neighboring lumens 26 are connected by and/or share adjacent walls. The adjacent walls may be formed by intertwined strands 40 that form a thickness extending between an outer diameter and an inner diameter for the lumens 26. The thickness of the walls and the corresponding diameters may vary based on the interlinked configuration, thickness of the strand 40 material, and/or layering of the strands 40 to form the tubular structure(s) 24. In certain aspects, the interjoined wall between adjacent lumens 26 may be thinner than the remainder of the wall of the tubular structure 24. The wall thickness of joining lumens 26 may be controlled by the number of carriers that repeatedly cross. For example, 32 carriers can provide more coverage in the center than 8. The tubular structures 24 and lumens 26 may be a substantially similar size or may be different sizes relative to one another. Further, the cross-section of the tubular structure 24 and/or the lumens 26 may vary along the length thereof.

The tubular structures 24 may be interlaced together, separate, or in combination thereof. For example, the tubular structures 24 may be interlaced to be joined along the length of the pledget 22. In certain aspects, the tubular structures 24 may adjust between being interlaced and bifurcated, to have joined segments (e.g., figure “8” shaped) and separated segments. The strands 40 may be carried along a predefined path that ensures the interlacing or separation. The carriers may interlace the strands 40 any practicable number of times over a predefined length, such as between 1 and 10 times or between 1 and 20 times. Typically, the interlacing may occur when one strand 40 passes over one strand 40, two strands 40 pass over one strand 40, two strands 40 pass over two strands 40, or any other practicable pattern.

In various examples, the tubular structure 24 may include a core/filled interior 48 or a hollow core (e.g., a hollow open lumen 26). When using a core 48, the suture 12 may be passed through the core 48. The core 48 may be interlinked with the tubular structure 24 or may be an insert positioned within the lumen 26. The core 48 may be compressible or may be at least semi-rigid to maintain the shape of the tube pledget 22.

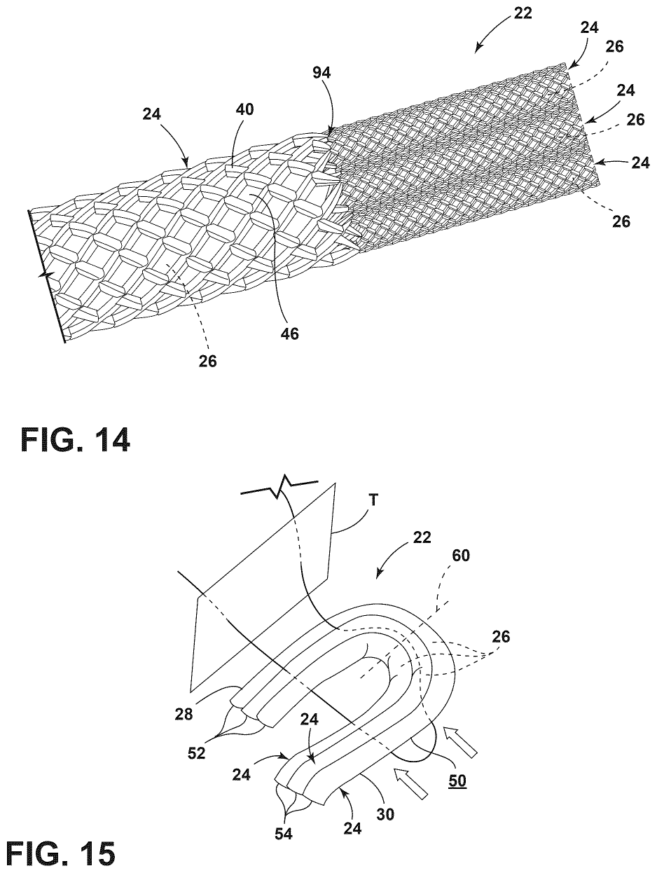

The tubular structure 24 may have one or more flatter or compressed segments 92 over a portion of the length or between adjacent tubular structures 24, such as the configuration illustrated in FIG. 13. The tubular structure 24 may also include transition sections 94 between different segments of the pledget 22, such as between different sized tubular structures 24, between different numbers of lumens 26, etc., such as the configuration illustrated in FIG. 14. For example, automatic braiding processes may be programmed to direct the paths of the corresponding strands 40 along one or more transition sections 94. Accordingly, the pledget 22 may transition between different numbers of lumens 26 and tubular structures 24.

Multiple sutures 12 may extend through the multiple lumens 26, respectively. This may form the suture assembly 10 having multiple sutures 12 and a single pledget 22. Additionally or alternatively, the suture 12 may extend through the pledget 22 in different ways. For example, as illustrated in FIG. 15, the pledget 22 may be folded to form a general “U” or “C” shape with the lumens 26 extending parallel with one another in a curved path. The end regions 28, 30 may be disposed adjacent to one another. At the end regions 28, 30, the suture 12 may extend through the tubular structure 24 and transversely across the lumens 26. Proximate to the centerline 60, the suture 12 may extend through the tubular structure 24 and into one of the lumens 26, along the lumen 26, and then out of the lumen 26 through the tubular structure 24. In certain aspects, the suture 12 may also extend transversely across one or more of the lumens 26. Such configurations may allow for increased movement of the pledget along the suture 12 while providing increased compression of the pledget 22 against the tissue T.

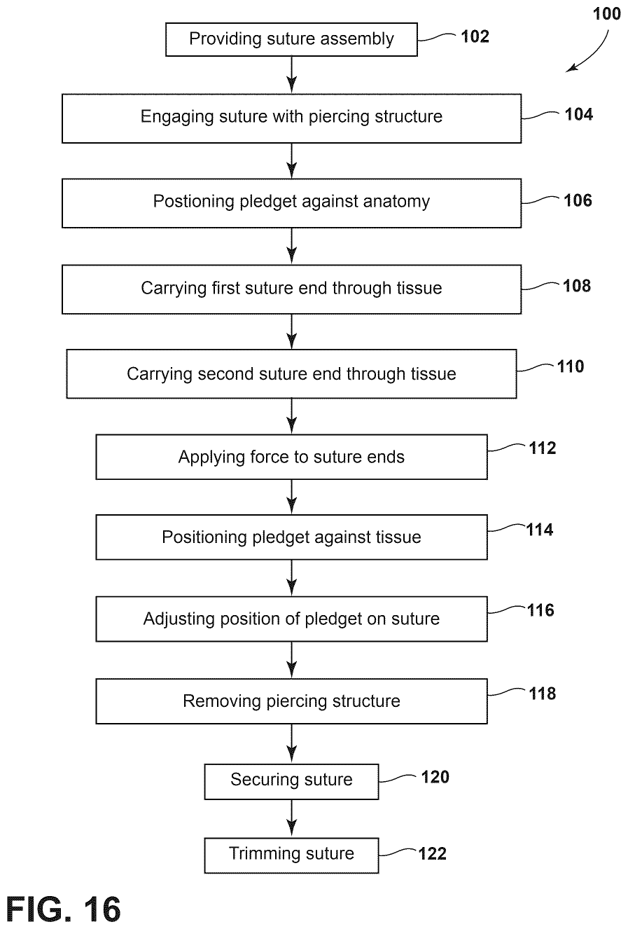

Referring to FIG. 16, as well as FIGS. 1-15, the suture assembly 10 may be utilized for performing one or more surgical procedures or methods (100), such as for replacing a valve or closing a cannulation site S. The suture assembly 10 may be provided (102). The suture assembly 10 may be pre-assembled. In other aspects, the suture assembly 10 may be assembled by the medical team. In such examples, the needles 18, 20 may be inserted through the tube pledget 22. Alternatively, the tube pledget 22 may be loaded onto the loading device 70, and the loading device 70 may assist with positioning the tube pledget 22 on the suture 12. The suture 12 may extend through the lumen 26 of the tube pledget 22 with the suture ends 14, 16 available for passing through the tissue T. The suture 12 may engage a piercing structure (104). The piercing structure may be the needles 18, 20 attached to the suture 12 or, alternatively, the suture passer or implant device engaging the suture ends 14, 16.

The tube pledget 22 may be positioned against the anatomy (106). In replacement valve V procedures, the tube pledget 22 may be positioned on the under side of the tissue T. In cannulation site S procedures, the tube pledget 22 may be positioned on an outer side of the tissue T.

The first suture end 14 may be passed through the tissue T (108). The first suture end 14 may also be passed through the replacement valve V. Additionally or alternatively, the first suture end 14 may be passed through the tissue T multiple times, such as along a portion of the cannulation site S. The second suture end 16 may be passed through the tissue T (110). The second suture end 16 may be passed through the replacement valve V and/or through the tissue T multiple times. A tensioning or pulling force F may be applied to the suture ends 14, 16 (112), which can pull the length of the suture 12 through the tissue T. The pulling force F can also pull or compress the tube pledget 22 against the tissue T (114). In response to the pulling force F being applied to the suture 12, the relationship between the suture 12 and the elongated tube pledget 22 can cause the suture 12 to unwind between the tube pledget 22 and the anatomy. Further, this relationship can reduce twisting or flipping of the tube pledget 22, maintaining the general cylindrical configuration or compressed cylindrical configuration of the tube pledget 22 against the tissue T.

The tube pledget 22 may be positioned to sit against the anatomy and between suture ends 14, 16 and, when used, the needles 18, 20. The position of the tube pledget 22 along the length of the suture 12 can be adjusted (116). The position may be changed by applying a pulling force F to one suture end 14, 16 and holding the tube pledget 22 or using the tissue T to hold the tube pledget 22. The piercing structure may be removed (118). In configurations with the needles 18, 20, the needles 18, 20 may be removed from the suture assembly 10. In configurations utilizing the suture passer or the implant device, the device may fully disengage from the suture 12. The suture 12 may then be secured (120), and excess suture 12 length may be trimmed and removed (122). The tube pledget 22 may be considered an extension or “arm” of the suture 12 that remains in contact with the tissue T of the anatomy (e.g., aorta, valve, atrium, and ventricle tissue T, etc.).

To manufacture the tube pledget 22, the strands 40 may be engaged with a respective number of carriers. The carriers may follow a predefined path to form the pledget 22, interlacing the strands 40 to form the tubular structure(s) 24 and the lumen(s) 26. The tubular structure(s) 24 may be formed of a predefined length. In certain aspects, the tubular structures 24 may be cut from the carriers at the predefined lengths for use in the suture assembly 10.

Additionally or alternatively, the tubular structure(s) 24 may be cut from the carriers and loaded onto a cutting frame. The cutting frame may be a predefined width and may include slots at intervals along the width. A select length of the tubular structure(s) 24 may be cut and inserted into the cutting frame. The cutting frame may have longitudinal grooves or channels that extend transversely through the slots. The tubular structure(s) 24 may extend through the channels along the length of the cutting frame. The tubular structure(s) 24 may be straightened with minimal pulling. The cutting frame may assist in retaining the tubular structure(s) 24 in the channels and may assist in locking the tubular structure(s) 24 in place relative to the cutting frame. Multiple, separate tubular structure(s) 24 may be held by a single cutting frame.

A lid or cover may be positioned over the tubular structure(s) 24 and the cutting frame. The cutting frame may be aligned with a cutting fixture. The cutting fixture may cut the tubular structure(s) 24 into predefined lengths, which may generally correspond to the spacing between slots in the cutting frame. As the tubular structure(s) 24 are cut into the pledgets 22, the ends of the pledgets 22 may be sealed as described herein to reduce fraying. The sutures 12 and/or the needles 18, 20 may then be added to the pledgets 22 to form the suture assemblies 10. The suture 12 may be threaded through the lumen 26, partially through the lumen 26, and/or across the walls of the tubular structure 24 as described herein.

Use of the present device may provide for a variety of advantages. For example, the elongated and narrower configuration of the tube pledget 22 may reduce the flipping or folding of the pledget 22 against the tissue T. This may result in increased efficiency during the surgical process and may reduce the size of the pledget against the tissue T. Additionally, the suture 12 may extend from the end regions 28, 30 of the pledget 22, which may assist with reducing the twisting of the pledget 22. Further, this relationship may result in automatic unwinding of the suture 12 in response to the pulling force F applied to the suture ends 14, 16. This may provide an increased seal and increased efficiency in the surgical procedure.

Moreover, in various aspects, the tubular structure 24 may extend circumferentially around the suture 12 to provide an extension of the suture 12 and reduce material that may be folded. Additionally or alternatively, the suture 12 may pass through the tubular structure 24 to provide increased compression or flattening of the tube pledget 22 against the tissue T. Also, the tube pledget 22 may not be sewn or locked into a fixed position on the suture 12 but may be passed/moved over the length of the suture 12 to act as an extension of the suture 12 that is in contact with the tissue T. Additionally, the hollow, cylindrical configuration with the lumen 26 may assist with moving the tube pledget 22 along the suture 12, reducing flipping and/or folding of the tube pledget 22, providing additional layers of the pledget to compress against the tissue T, and providing a better seal. Additional benefits or advantages may be realized and/or achieved.

The device disclosed herein is further summarized in the following paragraphs and is further characterized by combinations of any and all various aspects described herein.

According to an aspect of the present disclosure, a suture assembly may include a suture having first and second suture ends. A first needle may be coupled to the first suture end. A second needle may be coupled to the second suture end. A tube pledget may be disposed along the suture between the first and second needles. The tube pledget may include a tubular structure defining a lumen. The suture may extend from a first end region of the tubular structure to the first needle and from a second end region of the tubular structure to the second needle.

According to an aspect of the present disclosure, a suture assembly may include a suture having first and second suture ends. A first needle may be coupled to the first suture end. A second needle may be coupled to the second suture end. A tube pledget may be disposed along the suture between the first and second needles. The tube pledget may include a tubular structure formed from interlacing strands and defines a lumen. The suture may extend through the lumen and through end openings at opposing ends of the tubular structure.

According to an aspect of the present disclosure, a suture assembly may include a suture having first and second suture ends. A first needle may be coupled to the first suture end. A second needle may be coupled to the second suture end. A tube pledget may be disposed along the suture between the first and second needles. The tube pledget may include a tubular structure formed from interlacing strands. The tubular structure may include an inner portion configured to engage tissue and an outer portion that collectively defines a lumen. The suture may extend through the inner portion of the tubular structure proximate to opposing ends of the tubular structure.

According to an aspect of the present disclosure, a method of performing a surgical procedure may include providing a suture assembly including a tube pledget extending along a length of a suture with the suture extending from end regions of the tube pledget; carrying a first suture end through tissue; carrying a second suture end through the tissue spaced from the first suture end; positioning the tube pledget against the tissue between the first and second suture ends; applying a force to the first and second suture ends; automatically unwinding the suture between the tube pledget and the tissue in response to the force applied to the first and second suture ends; and compressing the tube pledget against the tissue in response to the force applied to the first and second suture ends.

According an aspect of the present disclosure, a method of manufacturing a suture assembly may include carrying strands along a predefined path; interlacing the strands to form at least one tubular structure defining at least one lumen; cutting a predefined length of the at least one tubular structure into at least one pledget having a predefined length; sealing ends of the at least one pledget; and coupling a suture within the at least one pledget, the suture extending at least partially within the lumen.

According to an aspect of the present disclosure, a suture may have first and second suture ends. A first needle may be coupled to the first suture end, and a second needle may be coupled to the second suture end. A tube pledget may be disposed along the suture between the first and second needles. The tube pledget may include a tubular structure formed from interlacing strands. The tubular structure may include an inner portion configured to engage tissue and an outer portion that collectively defines a lumen. The suture may extend through the lumen between the first and second needles. The suture may extend from a first end region of the tubular structure to the first needle and from a second end region of the tubular structure to the second needle.

According to any preceding aspect of the present disclosure, first and second end regions of a tubular structure may define first and second end openings of a lumen, respectively.

According to any preceding aspect of the present disclosure, a suture may extend through a lumen and may extend out of first and second end openings.

According to any preceding aspect of the present disclosure, a tubular structure may be free of a suture passing therethrough.

According to any preceding aspect of the present disclosure, a tube pledget may be configured to move along a suture between first and second needles.

According to any preceding aspect of the present disclosure, a tubular structure may be constructed of interlacing strands.

According to any preceding aspect of the present disclosure, a first set of interlacing strands may extend parallel to one another in a helical pattern in a first direction. A second set of interlacing strands may extend parallel to one another in a helical pattern in a second direction.

According to any preceding aspect of the present disclosure, a second set of interlacing strands may extend through a first set of interlacing strands to form a tubular structure.

According to any preceding aspect of the present disclosure, a tubular structure may include openings defined by interlacing strands.

According to any preceding aspect of the present disclosure, a tube pledget may include between about eight and about sixty-four strands.

According to any preceding aspect of the present disclosure, a suture may extend between adjacent strands of interlacing strands and transversely across a lumen.

According to any preceding aspect of the present disclosure, a suture may pass through a tubular structure at a first location proximate to a first end region of the tubular structure and at a second location proximate to a second end region of the tubular structure.

According to any preceding aspect of the present disclosure, a suture may extend through a lumen between first and second locations.

According to any preceding aspect of the present disclosure, a tubular structure may extend circumferentially around a suture.

According to any preceding aspect of the present disclosure, a suture may extend along an outer surface of a tubular structure between first and second locations.

According to any preceding aspect of the present disclosure, a tubular structure may be configured to compress to reduce a size of a lumen in response to an applied force on first and second needles to pull a tubular structure against tissue.

According to any preceding aspect of the present disclosure, a tube pledget may be constructed of at least one of polyester and polytetrafluoroethylene.

According to any preceding aspect of the present disclosure, a tube pledget may have a length between about 3 mm and about 36 mm.

According to any preceding aspect of the present disclosure, a tube pledget may have a diameter of less than about 2 mm.

According to any preceding aspect of the present disclosure, a tube pledget may be elongated with a length greater than a width, and the length may extend along a suture.

According to any preceding aspect of the present disclosure, a suture may extend through an outer portion and across a lumen proximate to opposing ends of a tubular structure.

According to any preceding aspect of the present disclosure, a suture may extend through a lumen between locations where the suture extends through an inner portion.

According to any preceding aspect of the present disclosure, a step of automatically unwinding a suture may include automatically rotating a tube pledget in response to a force applied to first and second suture ends.

According to any preceding aspect of the present disclosure, a method may include adjusting a position of a tube pledget along a length of a suture.

According to any preceding aspect of the present disclosure, a step of compressing a tube pledget against tissue may include applying force to end regions of the tube pledget and reducing folding of the tube pledget in response to the force applied to the end regions.

According to any preceding aspect of the present disclosure, a step of providing a suture assembly may include positioning a suture through a lumen of a tube pledget.

According to any preceding aspect of the present disclosure, a step of providing the suture assembly may include carrying first and second suture ends through a tubular structure of a tube pledget and transversely across a lumen.

According to any preceding aspect of the present disclosure, a step of providing a suture assembly may include providing the suture assembly with first and second needles coupled with first and second suture ends, respectively, and a tube pledget between the first and second needles.

According to any preceding aspect of the present disclosure, a step of carrying a first suture end through tissue may include carrying the first suture end through the tissue with a first needle, and a step of carrying a second suture end through the tissue may include carrying the second suture end through the tissue with a second needle.

According to any preceding aspect of the present disclosure, a method may include carrying a first suture end through a replacement valve, carrying the second suture end through the replacement valve, compressing the replacement valve against the tissue, and securing the suture.

According to any preceding aspect of the present disclosure, a step of compressing a tube pledget against tissue may include compressing the tube pledget against an under side of the tissue.

According to any preceding aspect of the present disclosure, a step of carrying a first suture end through tissue may include carrying the first suture end through the tissue at multiple locations around an incision, and a step of carrying a second suture end through the tissue may include carrying the second suture end through the tissue at multiple locations around the incision.

According to any preceding aspect of the present disclosure, a step of compressing a tube pledget against a tissue may include compressing a tube pledget against an outer side of the tissue.

According to any preceding aspect of the present disclosure, a method may include pulling tissue with a suture around an incision and closing the incision in response to the force applied to first and second suture ends.

According to any preceding aspect of the present disclosure, a step of cutting a predefined length and a step of sealing ends may be performed simultaneously. A step of cutting a predefined length and a step of sealing ends may be performed via at least one of heat cutting, laser cutting, or ultrasonic cutting.

According to any preceding aspect of the present disclosure, a step of sealing ends may be performed via adhesive end bonding, including at least one of cyanoacrylate, epoxy, and/or UV cure.

According to any preceding aspect of the present disclosure, a method may include coating a pledget with a medical-grade polymer.

According to any preceding aspect of the present disclosure, a method may include coupling needles to ends of a suture.

According to any preceding aspect of the present disclosure, a method may include loading a suture assembly on a loading device.

According to any preceding aspect of the present disclosure, a pledget may be absorbable.

According to any preceding aspect of the present disclosure, a kit may include a suture assembly pre-loaded or pre-coupled with a loading device.

According to any preceding aspect of the present disclosure, a loading device may be a predefined monofilament loop.

According to any preceding aspect of the present disclosure, a suture may extend through a lumen and through end openings at first and second end regions of a tubular structure.

According to any preceding aspect of the present disclosure, a tube pledget may have a diameter of less than about 4 mm.

According to any preceding aspect of the present disclosure, a tube pledget may be absorbable.

According to any preceding aspect of the present disclosure, a core may be disposed within a lumen of a tube pledget.

According to any preceding aspect of the present disclosure, a tube pledget may be constructed of an absorbable material.

According to any preceding aspect of the present disclosure, material density or thickness of a pledget may be between about 10 dtex and about 1000 dtex.

According to any preceding aspect of the present disclosure, a pledget may be constructed of strands made of one or more of: polyester, polypropylene, nylon, polybutester, silk, plain gut, chromic gut, polyglactin, poliglecaprone, PDS (polydioxanone), polydioxanone polyglycolic acid (e.g., Dexon®), vicryl, PTFE, monocryl, barbed suture, and/or stainless steel wire.

According to any preceding aspect of the present disclosure, a wall thickness of a pledget may be about 1 mm or less.

According to any preceding aspect of the present disclosure, a diameter of a pledget may be less than about 4 mm.

According to any preceding aspect of the present disclosure, a pledget may be constructed of strands having different thicknesses.

According to any preceding aspect of the present disclosure, a pledget may have ends sealed via at least one of heat cutting, laser cutting, ultrasonic cutting, adhesive end bonding, and/or being coated.

According to any preceding aspect of the present disclosure, a securing distance at ends of a pledget may be less than about 2 mm.

According to any preceding aspect of the present disclosure, a suture may be a 0-0 suture, a 2-0 suture, a 3-0 suture, a 4-0 suture, or a 5-0 suture.

According to any preceding aspect of the present disclosure, a tube pledget may include a tubular structure formed from interlacing strands.

According to any preceding aspect of the present disclosure, a suture may extend through a lumen between first and second needles, and the suture may extend from a first end region of a tubular structure to the first needle and from a second end region of the tubular structure to the second needle.

According to any preceding aspect of the present disclosure, a tubular structure includes sealed ends, an inner portion configured to engage tissue, an outer portion, and defines a lumen collectively defined by the inner and outer portions.

It will be understood that any described processes or steps within described processes may be combined with other disclosed processes or steps to form structures within the scope of the present device. The exemplary structures and processes disclosed herein are for illustrative purposes and are not to be construed as limiting.

As used herein, words of approximation such as, without limitation, “approximately,” “substantially,” or “about” refer to a condition that when so modified is understood to not necessarily be absolute or perfect but would be considered close enough to those of ordinary skill in the art to warrant designating the condition as being present. Further, the lack of such modifying terms does not otherwise require strict interpretation of the corresponding value or property. Instead, the extent to which the associated interpretation varies will depend on how great a change can be instituted and still have one of ordinary skill in the art recognize the modified feature as having the required characteristics or capabilities of the unmodified feature. Such determinations may vary considerably depending on the technological field based on the corresponding equivalency associated with the described operation or property. In general, but subject to the preceding discussion, a numerical value herein that is modified by a word of approximation such as approximately,” “substantially,” or “about” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. A suture assembly, comprising:
a suture having first and second suture ends;
a first needle coupled to the first suture end;
a second needle coupled to the second suture end; and
a tube pledget disposed along the suture between the first and second needles, wherein the tube pledget includes a tubular structure defining a lumen, and wherein the suture extends from a first end region of the tubular structure to the first needle and from a second end region of the tubular structure to the second needle.

2. The suture assembly of claim 1, wherein the first and second end regions of the tubular structure define first and second end openings of the lumen, respectively, and wherein the suture extends through the lumen and extends out of the first and second end openings.

3. The suture assembly of claim 2, wherein the tubular structure is free of the suture passing therethrough.

4. The suture assembly of claim 1, wherein the tube pledget is configured to move along the suture between the first and second needles.

5. The suture assembly of claim 1, wherein the tubular structure is constructed of interlacing strands.

6. The suture assembly of claim 5, wherein a first set of the interlacing strands extends parallel to one another in a helical pattern in a first direction, and wherein a second set of the interlacing strands extends parallel to one another in a helical pattern in a second direction.

7. The suture assembly of claim 6, wherein the second set extends through the first set to form the tubular structure.

8. The suture assembly of claim 5, wherein the tubular structure includes openings defined by the interlacing strands.

9. The suture assembly of claim 5, wherein the suture extends between adjacent strands of the interlacing strands and transversely across the lumen.

10. The suture assembly of claim 1, wherein the suture passes through the tubular structure at a first location proximate to the first end region of the tubular structure and at a second location proximate to the second end region of the tubular structure.

11. The suture assembly of claim 10, wherein the suture extends through the lumen between the first and second locations.

12. The suture assembly of claim 10, wherein the suture extends along an outer surface of the tubular structure between the first and second locations.

13. The suture assembly of claim 1, wherein the tubular structure extends circumferentially around the suture.

14. The suture assembly of claim 1, wherein the tube pledget is constructed of an absorbable material.

15. A suture assembly, comprising:
a suture having first and second suture ends;
a first needle coupled to the first suture end;
a second needle coupled to the second suture end; and
a tube pledget disposed along the suture between the first and second needles, wherein the tube pledget includes a tubular structure formed from interlacing strands, and wherein the tubular structure includes sealed ends, an inner portion configured to engage tissue, an outer portion, and a lumen defined by the inner and outer portions, and wherein the suture extends through the lumen between the first and second needles, and further wherein the suture extends from a first end region of the tubular structure to the first needle and from a second end region of the tubular structure to the second needle.

16. The suture assembly of claim 15, wherein the suture extends through the lumen and through end openings at the first and second end regions of the tubular structure.

17. The suture assembly of claim 15, wherein the suture extends through the inner portion of the tubular structure at the first and second end regions of the tubular structure.

18. The suture assembly of claim 17, wherein the suture extends through the outer portion and across the lumen proximate to the first and second end regions of the tubular structure.

19. The suture assembly of claim 17, wherein the suture extends through the lumen between locations where the suture extends through the inner portion.

20. A method of performing a surgical procedure, comprising:
providing a suture assembly including a tube pledget extending along a length of a suture with the suture extending from end regions of the tube pledget;
carrying a first suture end through tissue;
carrying a second suture end through the tissue spaced from the first suture end;
positioning the tube pledget against the tissue between the first and second suture ends;
applying a force to the first and second suture ends;
automatically unwinding the suture between the tube pledget and the tissue in response to the force applied to the first and second suture ends; and
compressing the tube pledget against the tissue in response to the force applied to the first and second suture ends.
